(19) United States
(12) Patent Application Publication
(10) Pub. No.: US 2009/0036658 A1
(43) Pub. Date: Feb. 5, 2009

(54) Highly efficient synthesis of alpha-O-galactosyl ceramides

(75) Inventors: Jacquelyn Gervay-Hague, Davis, CA (US); Wenjun Du, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/817,541

(22) PCT Filed: Mar. 3, 2006
(86) PCT No.: PCT/US06/07584

§ 371 Date: May 13, 2008

Related U.S. Application Data
(60) Provisional application No. 60/658,936, filed on Mar. 4, 2005.

(51) Int. Cl. C07H 15/08

(52) U.S. Cl. 536/17.9

(57) ABSTRACT

A method for the production of a-O-galactosyl ceramide precursor is demonstrated. The method involves the reaction of galactosyl iodide with a sphingosine derivative or phytosphingosine derivative in the presence of a quaternary ammonium iodide salt to prepare the a-O-galactosyl ceramide precursor in the a-anomer form. The a-O-galactosyl ceramide is then prepared by reaction with a suitable fatty acid or fatty acid derivative.

### CROSS-REFERENCES TO RELATED APPLICATIONS

This application claims priority to U.S. Provisional Application No. 60/658,936, filed Mar. 4, 2005.

### STATEMENT AS TO RIGHTS TO INVENTIONS MADE UNDER FEDERALLY SPONSORED RESEARCH OR DEVELOPMENT

This work was supported by National Science Foundation CHE-0196482. NSF CRIF program (CHE-9808183), NSF Grant OSTI 97-24412, and NIH Grant RR11973 provided funding for the NMR spectrometers used on this project. The govenment may have rights in this patent.

### BACKGROUND OF THE INVENTION

Since the discovery of α-O-galactosyl ceramides from marine sponge in 1993, (Natori, N. et al., Tetrahedron Lett. 34:5591-5592 (1993)) several studies of KRN7000 (1) and its derivatives have been reported. These investigations have revealed that tumor-associated cell-surface glycoproteins such as CD1d present exogenous lipids to natural killer T (NKT) cells causing the release of chemokines that regulate immune response to cancer ((a) Brutkiewicz, R. R. et al., Crit. Rev. Oncol./Hematol. 41:287-298 (2002); (b) Brutkiewicz, R. R. et al., Crit. Rev. Immunol. 23:403-419 (2003); (c) Zhou, D. et al., Science, 303:523-527 (2004). The potent immunostimulatory activities of lipid antigens such as KRN7000 have led to the development of anticancer chemotherapeutics that are currently in clinical trials (Crul, M. et al., Cancer Chemother, Pharmacol. 49:287-293 (2002)). Moreover, α-O-galactosyl ceramide analogs have provided important tools for elucidating signal transduction pathways involved in CD1d-mediated antigen presentation (Lin, Y. et al., Cancer Cell, submitted).

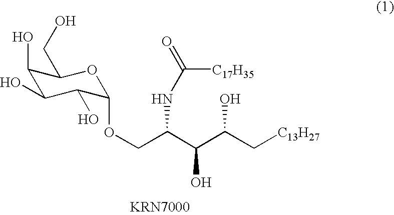

Several syntheses of KRN7000 analogs have been reported. However, current glycosidation methods suffer from relatively low yields and sometimes poor α/β selectivities. Galactosyl fluoride and trichloroacetimidate donors have been most successfully employed in the formation of et βO-glycosidic linkages. Yields for the fluoride typically range between 30-60% and are complicated by the formation of the β anomer, which is difficult to separate ((a) Morita, M. et al., J. Med. Chem. 38:2176-2187 (1995); (b) Takikawa, H. et al., 54:3141-3150 (1998); (c) Barbieri, L. et al., Eur. J. Org. Chem. 468-473 (2004); (d) Zhou, X.-T. et al., Org. Lett., 4:1267-1270 (2002)). Stereoselectivity can be improved with the use of galactosyl trichloroacetimidates ((a) Plettenburg, O. et al., J. Org. Chem. 67:4559-4564 (2002); (b) Kim, S. et al., Synthesis, 6:847-850 (2004)), but the reaction requires portionwise addition of both the donor and acceptor, otherwise yields drop and approximately one half of the unreacted acceptor is recovered (Fiegueroa-Perez, S. et al., Carbohydr. Chem. 328:95-102 (2000)). Thioglycosides ((a) Plettenburg, O. et al., J. Org. Chem. 67:4559-4564 (2002); (b) Kim, S. et al., Synthesis, 6:847-850 (2004)) and glycosyl bromides (Goff, R. D. et al., J. Am. Chem. Soc. 126:13602-13603 (2004)) have also been utilized, and while relatively easy to employ, both yields and stereoselectivities are compromised with these donors.

Other work shows that glycosyl bromides proceed via an in situ anomerization from the α-anomer to a more reactive β-anomer thereby allowing S_{N}2-like displacement to give exclusively the α-glycoside (Lemieux, R. U. et al., Am. Chem. Soc. 97:4056 (1975)). Surprisingly, we have discovered a remarkably efficient syntheses of α-O-galactosyl ceramides using galactosyl iodide donors in combination with a quaternary ammonium iodide salt. Conversion of the α-iodide to the more reactive β-iodide is orders of magnitude faster, proceeding quantitatively, with exclusive delivery of the α anomer. The combination of the galactosyl iodide and sphingosine derivative or phytosphingosine derivative with tetrabutylammonium iodide (TBAI) as a promoter, results in glycosidation of the sphingosine and phytosphingosine derivative in over 90% yield giving exclusively the α-O-galactosyl linkage.

### BRIEF SUMMARY OF THE INVENTION

In one aspect, the present invention provides a method of producing an α-O-galactosyl ceramide precursor comprising the step of contacting a galactosyl iodide with a quaternary ammonium iodide salt and a sphingosine derivative or a phytosphingosine derivative under conditions sufficient to produce an α-O-galactosyl ceramide precursor.

In another embodiment, the present invention provides a method of producing an α-O-galactosyl ceramide precursor wherein the galactosyl iodide has the formula:

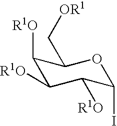

wherein each R¹ is an independently selected protecting group. In a further embodiment, each R¹ is a benzyl group. In still another embodiment, each R¹ is a tri-methyl silyl group.

In other embodiments, the present invention provides that the quaternary ammonium iodide salt is tetra-butylammonium iodide.

In a further embodiment, the present invention provides that the sphingosine derivative has the formula:

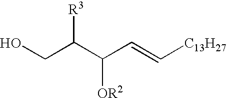

wherein R² is a member selected from the group consisting of a protecting group and H; and R³ is a member selected from the group consisting of an amine, an amide and an azide. In another embodiment, R² is a para-methoxybenzyl group. In still another embodiment, R² is tri-methyl silyl group. In yet another embodiment, R³ is an azide.

In other embodiments, the present invention provides that the phytosphingosine derivative has the formula:

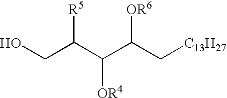

wherein R⁴ and R⁶ are each independently a member selected from the group consisting of a protecting group and H; and R⁵ is a member selected from the group consisting of an amine, an amide and an azide. In still other embodiments, R⁴ and R⁶ are each para-methoxybenzyl. In a farther embodiment, R⁵ is an azide.

In another embodiment, the present invention provides a method of producing an α-O-galactosyl ceramide precursor where the α-O-galactosyl ceramide precursor is prepared in at least 80% yield.

In another aspect, the present invention provides a method of producing an α-O-galactosyl ceramide comprising a first step of contacting a galactosyl iodide with a quaternary ammonium iodide salt and a sphingosine derivative or a phytosphingosine derivative under conditions sufficient to produce an α-O-galactosyl ceramide precursor. The second step involves contacting the α-galactosyl ceramide precursor with a fatty acid or fatty acid derivative under conditions appropriate to produce an α-O-galactosyl ceramide.

In other embodiments, the present invention provides a method where the galactosyl iodide has the formula:

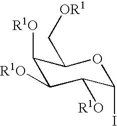

wherein each R¹ is an independently selected protecting group. In another embodiment, each R¹ is a benzyl group or a tri-methyl silyl group.

In another embodiment, the present invention provides a method where the quaternary ammonium iodide salt is tetra-butylammonium iodide.

In a further embodiment, the present invention provides a method where the sphingosine derivative has the formula:

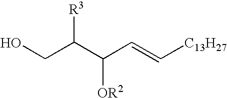

wherein R¹ is a para-methoxybenzyl group or a tri-methyl silyl group; and R³ is an azide.

In other embodiments, the present invention provides a method where the phytosphingosine derivative has the formula:

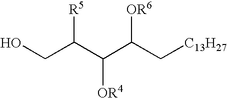

wherein R⁴ and R⁶ are each para-methoxybenzyl; and R⁵ is an azide.

In another embodiment, the present invention provides a method where the fatty acid is stearic acid.

### DETAILED DESCRIPTION OF THE INVENTION

### I. Definitions

As used herein, the term “contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

As used herein, the term “protecting group” refers to a compound that renders a functional group unreactive, but is also removable so as to restore the functional group to its original state. Such protecting groups are well known to one of ordinary skill in the art and include compounds that are disclosed in “Protective Groups in Organic Synthesis”, 3rd edition, T. W. Greene and P. G. M. Wuts, John Wiley & Sons, New York, 1999, which is incorporated herein by reference in its entirety.

As used herein, the term “fatty acid” refers to a carboxylic acid having an aliphatic tail, typically from 4 to 30 carbon atoms long. Fatty acids can be saturated, mono-unsaturated or poly-unsaturated. Examples of fatty acids useful in the present invention, include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). One of skill in the art will appreciate that other fatty acids are useful in the present invention.

As used herein, the term “fatty acid derivative” refers to a fatty acid that has been modified to include an additional functional group or to incorporate a protecting group. A fatty acid derivative is characterized by having an aliphatic chain of between 4 and 30 carbon atoms, with a carboxylic acid at one terminus of the chain. Examples of fatty acid derivatives include those where the carboxylic acid has been modified so as to make the carboxylic acid more reactive or less reactive. A less reactive fatty acid derivative can be obtained via addition of a protecting group. A more reactive fatty acid derivative can be obtained by activation of the carboxylic acid, such as by conversion of the acid to an acid chloride. Additional fatty acid derivatives include those where the aliphatic chain is branched, or contains additional functionality.

As used herein, the term “ceramide precursor” refers to a compound that is between one and four synthetic steps away from synthesizing a ceramide. The ceramide precursors of the present invention are typically two synthetic steps from a ceramide. For example, a ceramide precursor of the instant invention can be the product resulting from reaction of a sphingosine derivative or phytosphingosine derivative with a galactosyl iodide. The ceramide can then be prepared by first deprotecting the amine on the sphingosine derivative or phytosphingosine derivative, followed by amine coupling with a fatty acid or fatty acid derivative. One of skill in the art will appreciate that other ceramide precursors are envisioned by the instant invention.

As used herein, the term “sphingosine derivative” refers to a sphingosine that has been modified to include an additional functional group or to incorporate a protecting group. Sphingosine is characterized by having an 18 carbon chain with hydroxyl groups at the 1 and 3 positions, an amine at the 2 position, and unsaturation at the 4 position. A sphingosine derivative can have any or all of the following derivations: additional functional groups (hydroxyl groups); be fully saturated; or have at least one of the functional groups protected with a protecting group. In addition, the sphingosine derivative can be linked to another molecule via one of the functional groups. Typical sphingosine derivatives of the present invention will have protecting groups on one or more of the functional groups of sphingosine. One of skill in the art will appreciate that other sphingosine derivatives are useful in the present invention.

As used herein, the term “phytosphingosine derivative” refers to a phytosphingosine that has been modified to include an additional functional group or to incorporate a protecting group. Phytosphingosine is characterized by having an 18 carbon chain with hydroxyl groups at the 1, 3 and 4 positions, an amine at the 2 position, and is fully saturated. A phytosphingosine derivative can have any or all of the following derivations: additional functional groups (hydroxyl or amino groups); unsaturation; or have at least one of the functional groups protected with a protecting group. In addition, the phytosphingosine derivative can be linked to another molecule via one of the functional groups. Typical phytosphingosine derivatives of the present invention will have protecting groups on one or more of the functional groups of phytosphingosine. One of skill in the art will appreciate that other phytosphingosine derivatives are useful in the present invention.

### II. Methods

The present invention provides a method of producing an α-O-galactosyl ceramide by first preparing an α-O-galactosyl ceramide precursor via coupling of a sphingosine derivative or a phytosphingosine derivative with a galactosyl iodide. The α-O-galactosyl ceramide precursor can then be derivatized to prepare the α-O-galactosyl ceramide via coupling of the α-O-galactosyl ceramide precursor with a fatty acid or fatty acid derivative.

### A. Preparation of α-O-galactosyl Ceramide Precursor

The α-galactosyl ceramide precursor of the present invention is produced by contacting a galactosyl iodide with a quaternary ammonium iodide salt and a sphingosine derivative or a phytosphingosine derivative under conditions sufficient to produce an α-O-galactosyl ceramide precursor.

The galactosyl iodide of the present invention is a galactose molecule where one of the hydroxy groups has been replaced with an iodine. Any of the hydroxy groups on galactose can be replaced by iodine. Preferably, the hydroxy group at the anomeric carbon is replaced with iodine. In one embodiment, the galactosyl iodide of the method of the present invention has the following formula, wherein each R¹ is a protecting group:

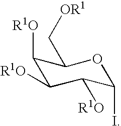

The protecting groups of the galactosyl iodide include any hydroxyl protecting group, including, but not limited to, benzyl, para-methoxybenzyl (PMB), triphenylmethyl (trityl), any tri-alkyl silyl group such as tri-methyl silyl (TMS), and ethers such as tetrahydropyranyl ether (THP) and methoxymethylether (MOM) groups. Protecting groups are selected for their stability during subsequent transformations and for their ease of removal. Other factors may influence the choice of protecting groups. Such protecting groups are well known to one of ordinary skill in the art and include compounds that are disclosed in “Protective Groups in Organic Synthesis”, 3rd edition, T. W. Greene and P. G. M. Wuts, John Wiley & Sons, New York, 1999.

Quaternary ammonium iodide salts useful in the method of the present invention include any tetra-alkyl ammonium iodide salt. Preferably, tetra-butyl ammonium iodide salt is used. One of skill in the art will appreciate that other quaternary ammonium iodide salts are useful in the methods of the present invention.

The sphingosine derivative useful in the method of the present invention has the following formula:

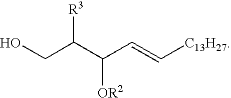

R² of the formula is a protecting group, such as those described above, or H, and R³ an amine, an amide or an azide. Protecting groups useful for R² include any hydroxy protecting groups such as those listed above for R¹. In some embodiments, R² is a para-methoxybenzyl group. In other embodiments, R² is a tri-methyl silyl group. In addition, R³ can be an azide.

The phytosphingosine derivative useful in the method of the present invention has the following formula:

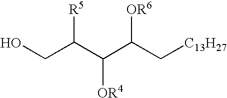

R⁴ and R⁶ of the formula can each independently be a protecting group or H. Protecting groups useful for R⁴ and R⁶ include any hydroxy protecting groups such as those listed above for R¹. R⁵ can be an amine, an amide or an azide. In some embodiments, R⁴ and R⁶ can be both a para-methoxybenzyl group. R⁵ can be an azide.

When the galactosyl iodide is contacted with the sphingosine derivative or phytosphingosine derivative in the presence of a quaternary ammonium iodide salt under the conditions of the present invention to produce an α-O-galactosyl ceramide precursor, the α-O-galactosyl ceramide precursor can be prepared in at least 40% yield. In other embodiments, the α-O-galactosyl ceramide precursor can be prepared in at least 60% yield. In still other embodiments, the α-O-galactosyl ceramide precursor can be prepared in at least 80% yield.

Preparation of an α-O-Galactosyl Ceramide Precursor of the Present Invention can proceed using a variety of solvents and reagents. Solvents useful for the preparation of α-O-galactosyl ceramide precursor of the present invention include, but are not limited to, aromatic solvents such as benzene and toluene. The preparation of the α-O-galactosyl ceramide precursor of the present invention can proceed at a variety of temperatures and times. Preparation of the α-O-galactosyl ceramide precursor of the present invention can be achieved over 0.5-16 hours at 40-100° C. Preferably, 0.5-4 hours at 50-80° C. is used. More preferably, 1-2 hours at 55-75° C. is used. One of skill in the art will appreciate that the time, temperature and solvent are dependent on each other, and changing one can require changing the others to prepare the α-O-galactosyl ceramide precursor of the present invention.

Other reagents useful in the preparation of the α-galactosyl ceramide precursor of the present invention include a non-nucleophilic base. The non-nucleophilic bases of the present invention can be tertiary bases. Nonnucleophilic bases useful in the present invention include, but are not limited to, di-isopropyl ethyl amine (DIPEA), tri-ethyl amine and quinuclidine. One of skill in the art will appreciate that other bases are useful in the present invention.

### B. Preparation of α-O-galactosyl Ceramide

The α-O-galactosyl ceramides of the present invention can be produced by contacting the α-O-galactosyl ceramide precursor prepared above, with a fatty acid or fatty acid derivative under conditions appropriate to produce an α-O-galactosyl ceramide.

The galactosyl iodide of the present invention is a galactose molecule where one of the hydroxy groups has been replaced with an iodine. Any of the hydroxy groups on galactose can be replaced by iodine. Preferably, the hydroxy group at the anomeric carbon is replaced with iodine. In one embodiment, the galactosyl iodide of the method of the present invention has the following formula, wherein each R¹ is a protecting group:

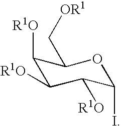

The protecting groups of the galactosyl iodide include any hydroxyl protecting group, including, but not limited to, benzyl, any tri-alkyl silyl group such as tri-methyl silyl, and ethers such as trityl, THP and MOM groups. Such protecting groups are well known to one of ordinary skill in the art and include compounds that are disclosed in “Protective Groups in Organic Synthesis”, 3rd edition, T. W. Greene and P. G. M. Wuts, John Wiley & Sons, New York, 1999.

Quaternary ammonium iodide salts useful in the method of the present invention include any tetra-alkyl ammonium iodide salt. Preferably, tetra-butyl ammonium iodide salt is used. One of skill in the art will appreciate that other quaternary ammonium iodide salts are useful in the methods of the present invention.

The sphingosine derivative useful in the method of the present invention has the following formula:

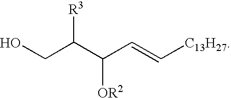

R² of the formula is a protecting group, such as those described above, or H, and R³ an amine, an amide or an azide. Protecting groups useful for R² include any hydroxy protecting groups such as those listed above for R¹. In some embodiments, R² is a para-methoxybenzyl group. In other embodiments, R² is a tri-methyl silyl group. In addition, R³ can be an azide.

The phytosphingosine derivative useful in the method of the present invention has the following formula:

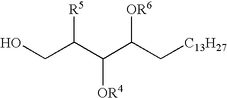

R⁴ and R⁶ of the formula can each independently be a protecting group or H. Protecting groups useful for R⁴ and R⁶ include any hydroxy protecting groups such as those listed above for R¹. R⁵ can be an amine, an amide or an azide. In some embodiments, R⁴ and R⁶ can be both a para-methoxybenzyl group. R⁵ can be an azide.

The fatty acids or fatty acid derivatives useful in the present invention include, but are not limited to, butyric acid (C4), caproic acid (C6), caprylic acid (C8), capric acid (C10), lauric acid (C12), myristic acid (C14), palmitic acid (C16), palmitoleic acid (C16), stearic acid (C18), oleic acid (C18), vaccenic acid (C18), linoleic acid (C18), alpha-linoleic acid (C18), gamma-linolenic acid (C18), arachidic acid (C20), gadoleic acid (C20), arachidonic acid (C20), eicosapentaenoic acid (C20), behenic acid (C22), erucic acid (C22), docosahexaenoic acid (C22), lignoceric acid (C24) and hexacosanoic acid (C26). The fatty acids useful in the present invention have at least four carbon atoms in the chain. Preferably, the fatty acids of the present invention have between 10 and 26 atoms in the chain. More preferably, the fatty acids have between 14 and 22 atoms in the chain. Most preferably, the fatty acids have between 16 and 20 atoms in the chain. In other instances, it is preferred that the fatty acids of the present invention have 26 atoms in the chain. The fatty acids of the present invention can be saturated, mono-unsaturated, or poly-unsaturated. Preferably, the fatty acids are saturated. Examples of fatty acid derivatives include those where the carboxylic acid has been modified so as to make the carboxylic acid more reactive or less reactive. A less reactive fatty acid derivative can be obtained via addition of a protecting group. A more reactive fatty acid derivative can be obtained by activation of the carboxylic acid, such as by conversion of the acid to an acid chloride. Additional fatty acid derivatives include those where the aliphatic chain is branched, or contains additional functionality. One of skill in the art will recognize that other fatty acids or fatty acid derivatives are useful in the present invention.

Preparation of an α-O-Galactosyl Ceramide of the Present Invention can Proceed using a variety of solvents and reagents. Solvents useful for the preparation of α-O-galactosyl ceramide of the present invention include, but are not limited to, amine-based solvents such as pyridine, tri-ethyl amine and N-methylpyrrolidinone (NMP), polar solvents such as methylene chloride, chloroform, tetrahydrofuran (THF), glyme, diglyme and ethyl acetate, as well as protic solvents such as ethanol, isopropanol, methanol, ethylene glycol and glycerol. The preparation of the α-O-galactosyl ceramide of the present invention can proceed at a variety of temperatures and times. Preparation of the α-O-galactosyl ceramide precursor of the present invention can be achieved over 1-36 hours at 0-50° C. Preferably, 1-24 hours at 20-40° C. is used. In some instance, 2-4 hours at room temperature is used. In other instances, 12-20 hours at room temperature is used. One of skill in the art will appreciate that the time, temperature and solvent are dependent on each other, and changing one can require changing the others to prepare the α-O-galactosyl ceramide of the present invention.

The coupling of the fatty acid or fatty acid derivative to the α-O-galactosyl ceramide precursor of the present invention can proceed via any amidation reaction known to one of skill in the art. Preferably, EDC coupling is used with DMAP to prepare the α-O-galactosyl ceramide of the present invention. Other coupling reactions are also useful and are known to one of skill in the art.

Additional steps in the production of the α-O-galactosyl ceramide can include removal of any remaining protecting groups. Removal of remaining protecting groups can be accomplished using standard methods known to one of skill in the art and will depend on the choice of protecting group and stability of the α-O-galactosyl ceramide to the reaction conditions (see also “Protective Groups in Organic Synthesis”, 3rd edition, T. W. Greene and P. G. M. Wuts, John Wiley & Sons, New York, 1999).

The following examples are provided by way of illustration only and not by way of limitation. Those of skill in the art will readily recognize a variety of non-critical parameters that can be changed or modified to yield essentially similar results.

### III. Examples

General. All reactions were conducted under a dried argon stream. Solvents (CH₂Cl₂ 99.8%, Benzene 99.8%) were purchased in capped DriSolv™ bottles and used without further purification and stored under argon. TMSI was stored at −15° C. under desiccated atmosphere. All other solvents and reagents were used without further purification. All glassware utilized was flame-dried before use. Glass-backed TLC plates (Silica Gel 60 with a 254 nm fluorescent indicator) were used without further manipulation and stored over desiccant. Developed TLC plates were visualized under a short-wave UV lamp, stained with an I₂—SiO₂ mixture, and/or by heating plates that were dipped in ammonium molybdate/cerium (IV) sulfate solution. Flash column chromatography (FCC) was performed using flash silica gel (32-63 μm) and employed a solvent polarity correlated with TLC mobility. Optical rotations were measured at 598 nm on a Jasco DIP-370 digital polarimeter using a 100 mm cell. NMR experiments were conducted on a Varian 400 MHz instrument using CDCl₃ (99.9% D) or C₅N₅D (99.9% D) as the solvent. Chemical shifts are relative to the deuterated solvent peak and are in parts per million (ppm). Low resolution mass spectra were acquired using a ThermoQuest Survey™ LC/MS instrument. High resolution mass spectra were obtained from a MALDI/FTMS instrument equipped with 7 Telsa magnets in positive mode in acetonitril/H₂O (9:1).

### EXAMPLE 1

### Preparation of a Sphingosine Derivative

A sphingosine derivative can be prepared as shown in Scheme 1. The amine is preferably protected as an azide for the glycosidation step, as amides diminish the nucleophilicity of the primary hydroxyl through hydrogen bond donation ((a) Schmidt, R. R. et al., Chem., Int. Ed. In Engl. 19:731 (1980); (b) Szabo, L. et al., Tetrahedron Lett. 32:585-588 (1991)). The primary alcohol was temporarily blocked with a trityl ether and the secondary alcohol was protected with an electron donating ether (p-methoxybenzyl (PMB)) to enhance the overall nucleophilicity of the acceptor alcohol. After deprotection of the trityl group with BF₃.OEt₂, a sphingosine acceptor (3) is available for glycosidation.

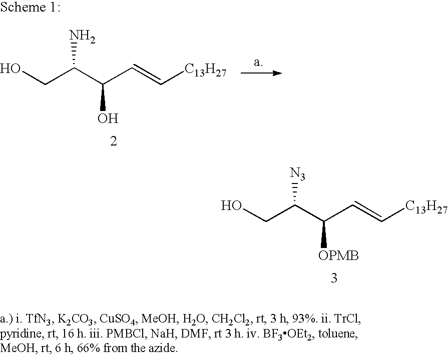

(2S,3R,4E)-2-Azido-3 para-methoxybenzyl-4-octadecence-1-ol (3). To a solution of the 2-azido-sphingosine (466 mg, 1.43 mmol) in pyridine (1 mL) and CH₂Cl₂ was added TrCl (440 mg, 1.58 mmol). The reaction mixture was stirred under argon for 24 hour. Solvent was evaporated and the crude product was dissolved in DMF (5 mL). NaH (57 mg, 2.36 mmol), followed by PMBCL (368 mg, 2.36 mmol) was added, the reaction mixture was stirred under argon for 2 hr. MeOH (5 mL) was added and solvent was evaporated. CH₂Cl₂ (10 mL) was added and the white precipitate was filtrated through Celite. The filtrate was collected and solvent was evaporated in vacuo. The crude product was dissolved in toluene (6 mL) and MeOH (2 mL). BF₃.OEt₂ (240 mg, 1.7 mmol) was added dropwise over a period time of 5 min. After 6 h reaction time, saturated NaHCO₃ solution (1 mL) was added and the solution was extracted with EtOAc (3×10 mL), the organic solution was combined and dried with anhydrous Na₂SO₄. Upon concentration in vacuo, FCC was applied to purify the compound to afford 3 as slightly yellow oil (421 mg, 66% from the 2-azido-sphingosine). [α]_{D}²⁵-46.7° (C=1.0, CH₂Cl₂). ¹H NMR (400 MHz, CDCl₃) δ 0.88 (t, J=6.8 Hz, 3H), 1.26-1.44 (m, 22H), 2.11 (dd, J=13.6, 6.8 Hz, 2H), 2.35 (t, J=6.0 Hz, 1H), 3.45 (dd, J=10.8, 6.0 Hz, 1H), 3.70 (m, 2H), 3.79 (s, 3H), 3.86 (dd, J=8.4, 6.0 Hz, 1H), 4.28 (d, J=11.2 Hz, 1H), 4.55 (d, J=11.2 Hz, 1H), 5.42 (dd, J=15.6, 8.4 Hz, 1H), 5.75 (dt, J=15.6, 6.8 Hz), 6.87 (dd, J=6.4, 2.0 Hz, 2H), 7.21 (dd, J=6.4, 2.0 Hz, 2H). ¹³C NMR (100 MHz, CDCl₃) δ 14.3, 22.9, 29.2, 29.4, 29.6, 29.7, 29.8, 29.87, 29.88, 32.1, 32.6, 55.4, 62.8, 66.2, 69.8, 80.4, 114.0, 126.4, 129.6, 130.0, 138.4, 159.4. ESIMS calcd for C₂₆H₄₃N₃O₃ [M+Na]⁺468.32, found: 468.71.

### EXAMPLE 2

### Preparation of α-O-galactosyl ceramide 6

The iodide donor (4) can be generated in situ from 2,3,4,6-O-benzyl galactosyl acetate according to a known procedure (Hadd, M. J. et al., Carbohydr. Res. 320:61-69 (1999)). Using TBAI as a promoter, 4 was contacted with the sphingosine derivative (3) to afford the α-glycoside (5) (Scheme 2). The azide was be reduced via a Staudinger reduction utilizing hydrogen sulfide in pyridine/water. The synthesis of 4-desoxy KRN7000 (6) was then completed by condensation of the amine with stearic acid followed by hydrogenation, resulting in global deprotection and concomitant reduction of the double bond.

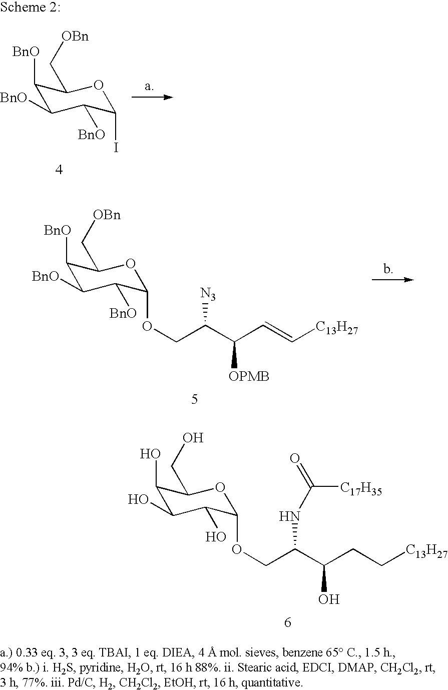

(2S,3R,4E)-2-Azido-3-O-para-methoxybenzyl-1-O-(2,3,4,6-tetra-O-benzyl-D-galactopyranosyl)-4-octadecene (5). To a solution of 1-O-acetyl-2,3,4,6-tetra-O-benzyl-D-galactopyranoside (290 mg, 0.50 mmol) in CH₂Cl₂ (3 mL) at 0° C. was added TMSI (100 mg, 0.50 mmol). The reaction mixture was stirred for at 0° C. for 20 min. The reaction was stopped by adding 3 mL of anhydrous toluene and azeotroped three times with toluene. The slightly yellow residue was dissolved in benzene (1 mL) and kept under argon. In a separate flask, molecular sieves (MS, 4 Å, 100 mg), TBAI (549 mg, 1.49 mmol), 3 (74 mg, 0.166 mmol) and DIPEA (64 mg, 0.50 mmol) was added into benzene (1 mL). The mixture was stirred under argon at 65° C. for 10 min. Upon dissolve of TBAI, the glycosyl iodide was cannulated into and the reaction mixture was stirred at 65° C. for 1.5 h. The reaction was stopped by adding EtOAc (10 mL) and cooled to 0° C., the white precipitate and MS was removed by filtration through Celite. The filtrate was washed with sat. Na₂S₂O₃ (aq) (2×10 mL) and brine, dried with anhydrous Na₂SO₄, concentrated in vacuo and the resulting residue was purified by FCC to afford 5 (159 mg, 94%) as clear oil. [α]_{D}²⁵+40.20 (C=0.5, CH₂Cl₂). ¹H NMR (400 MHz, CDCl₃) δ 0.89 (t, J=6.8 Hz, 3H), 1.28-1.65 (m, 22H), 2.09 (dd, J=13.6, 6.8 Hz, 2H), 3.53 (d, J=6.4 Hz, 2H), 3.59 (dd, J=12.4, 6.8 Hz, 2H), 3.78-3.3.82 (m, 4H), 3.91 (dd, J=8.4, 5.2 Hz, 1H), 3.96-3.99 (m, 3H), 4.06 (dd, J=9.6, 3.6 Hz, 1H), 4.22 (d, J=11.6 Hz, 1H), 4.41 (d, J=12.0 Hz, 1H), 4.48 (dd, J=11.2, 3.2 Hz, 2H), 4.58 (d, J=11.6 Hz, 1H), 4.69 (d, J=12.0 Hz, 1H), 4.74 (d, J=11.6 Hz, 1H), 4.82 (d, J=11.6 Hz, 1H), 4.85 (d, J=12.0 Hz, 1H), 4.90 (d, J=3.6 Hz, 1H), 4.95 (d, J=11.6 Hz, 1H), 5.39 (dd, J=15.6, 8.8 Hz, 1H), 5.71 (dt, J=15.6, 6.8 Hz, 1H), 6.85 (d, J=8.8 Hz, 2H), 7.20 (d, J=8.8 Hz, 2H), 7.25-7.40 (m, 20H). ¹³C NMR (100 MHz, CDCl₃) δ 14.4, 22.9, 29.3, 29.4, 29.6, 29.7, 29.9, 29.93, 31.8, 32.2, 32.6, 55.4, 64.4, 68.1, 69.2, 69.8, 69.9, 73.3, 73.4, 73.7, 75.0, 75.3, 76.7, 78.9, 79.2, 98.9, 113.9, 126.4, 127.67, 127.69, 127.7, 127.8, 127.9, 127.92, 127.98, 128.4, 128.45, 128.5, 128.53, 128.6, 129.4, 130.5, 138.1, 138.3, 138.8, 138.9, 139.0, 159.3. ESIMS calcd for C₆₀H₇₇N₃O₈ [M+Na]⁺990.52, found: 990.56.

(2S,3R,4E)-2-Amino-3-O-para-methoxybenzyl-1-O-(2,3,4,6-tetra-O-benzyl-α-D-galactopyranosyl)-4-octadecene (Amino derivative). 5 (60 mg, 0.62 mmol) was dissolved in pyridine (6 mL) and H₂O (6 mL). H₂S, generated from Na₂S and 3M H₂SO₄, was bubbled into over a period of 1 h. The reaction was kept sealed overnight. The solvent was removed in vacuo and the resulting residue was purified by FCC to afford desired product (51 mg, 88%) as white powder. ¹H NMR (400 MHz, CDCl₃) δ 0.88 (t, J=6.8 Hz, 3H), 1.26-1.38 (m, 22H), 2.09 (dd, J=13.6, 6.8 Hz, 2H), 3.06 (ddd, J=9.6, 6.8, 3.6 Hz, 1H) 3.36 (dd, J=9.6, 8.0 Hz, 1H), 3.52 (m, 2H), 3.66 (t, J=7.6 Hz, 1H), 3.77 (s, 3H), 3.86 (dd, J=9.6, 3.6 Hz, 1H), 3.93 (m, 3H), 4.03 (dd, J=9.6, 3.6 Hz, 1H), 4.19 (d, J=11.2 Hz, 1H), 4.39 (d, J=12.0 Hz, 1H), 4.46 (d, J=11.2 Hz, 1H), 4.47 (d, J=12.0 Hz, 1H), 4.56 (d, J=11.2 Hz, 1H), 4.65 (d, J=11.6 Hz, 1H), 4.71-4.82 (m, 3H), 4.89 (d, J=3.6 Hz, 1H), 4.92 (d, J=11.2 Hz, 1H), 5.35 (dd, J=15.6, 8.4 Hz, 1H), 5.70 (dt, J=15.6, 6.8 Hz, 1H), 6.82 (d, J=8.4 Hz, 2H), 7.19 (d, J=8.4 Hz, 2H), 7.25-7.40 (m, 20H). ¹³C NMR (100 MHz, CDCl₃) δ 14.4, 22.9, 29.5, 29.6, 29.7, 29.9, 30.0, 32.2, 32.7, 54.8, 55.5, 69.2, 69.6, 69.8, 71.0, 73.1, 73.4, 73.7, 75.0, 75.1, 76.9, 79.2, 81.5, 99.0, 113.9, 127.5, 127.66, 127.68, 127.8, 127.9, 127.99, 128.0 128.4, 128.44, 128.5, 128.56, 128.6, 129.5, 130.9, 137.6, 138.2, 138.8, 138.9, 139.0, 159.3. ESIMS calcd for C₆₀H₇₉NO₈ [M+H]⁺ 942.59, found: 942.51.

(2S,3R,4E)-3-O-para-Methoxybenzyl-1-O-(2,3,4,6-tetra-O-benzyl)-α-D-galactopyranosyl)-2-(N-octadecanosylamino)-4-octadecene (Amide derivative). To a stirred solution of the amino derivative (50 mg, 0.053 mmol) in CH₂Cl₂ (2 mL), EDCI (24 mg, 0.122 mmol), DMAP (catalytic amount) and stearic acid (31 mg, 0.126 mmol) were added. The reaction mixture was stirred under argon for 3 h. Solvent was removed in vacuo and the resulting residue was purified by FCC to afford the desired product (49 mg, 77%) as a white powder. ¹H NMR (400 MHz, CDCl₃) δ 0.87 (t, J=6.8 Hz, 6H), 1.26-1.67 (m, 52H), 2.01 (m, 4H), 3.35 (dd, J=9.6, 6.4, 1H), 3.46 (dd, J=9.6, 6.4 Hz, 1H), 3.62 (dd, J=11.2, 3.6 Hz, 1H), 3.70 (s, 3H), 3.77 (m, 2H), 3.84 (m, 2H), 3.98 (dd, J=9.6, 3.6 Hz, 1H), 4.04-4.11 (m, 2H), 4.18 (d, J=11.6 Hz, 1H), 4.36 (d, J=11.6 Hz, 1H), 4.46 (d, J=11.6 Hz, 1H), 4.48 (d, J=11.6 Hz, 1H), 4.55 (d, J=11.6 Hz, 1H), 4.59 (d, J=11.6 Hz, 1H), 4.72-4.82 (m. 4H), 4.90 (d, J=11.6 Hz, 1H), 5.35 (dd, J=15.6, 7.6 Hz, 1H), 5.70 (dt, J=15.6, 6.8 Hz, 1H), 6.08 (d, J=9.2 Hz, 1H), 6.77 (d, J=8.4 Hz, 2H), 7.18 (d, J=8.4 Hz, 2H), 7.25-7.40 (m, 20H). ¹³C NMR (100 MHz, CDCl₃) δ 14.38, 22.9, 25.9, 29.6, 29.61, 29.62, 29.65, 29.7, 29.8, 29.84, 29.9, 29.94, 30.0, 32.1, 32.6, 37.1, 52.6, 55.4, 69.0, 69.4, 69.7, 69.9, 73.1, 73.6, 73.7, 74.9, 75.0, 76.9, 78.7, 79.0, 99.7, 113.9, 127.7, 127.8, 127.86, 127.88, 127.9, 128.0, 128.1, 128.56, 128.58, 128.6, 129.7, 129.8, 130.7, 137.0, 137.9, 138.7, 138.8, 138.9, 159.3, 172.8. ESIMS calcd for C₇₈H₁₁₃NO₉ [M+Na]⁺1230.83, found: 1230.88.

(2S,3R)-1-O-(α-D-Galactopyranosyl)-2-(N-octadecanosylamino)-1,3-octadecenediol (6). The amide derivative (36 mg, 0.030 mmol) and Pd/C (36 mg, 100% w/w) was suspended in a solution of CH₂Cl₂ (0.5 mL) and EtOH (3 mL). The reaction mixture was put on a hydrogenation shaker with a pressure of 65 psi. After 16 h reaction, the mixture was filtered through Celite and washed with copious solvent of EtOH/CH₂Cl₂ (90/10, v/v). The solvent was removed in vacuo and the resulting residue was added with CH₂Cl₂ (5 mL) to silica gel (20 mg). The solution was sonicated and solvent was removed in vacuo. The resulting silica powder was loaded into a column and purified with FCC to afford 6 (23 mg, quant.) as a slightly yellow powder. ¹H NMR (400 MHz, C₅D₅N) δ 0.88 (t, J=6.8 Hz, 6H), 1.26-1.39 (m, 53H), 1.58 (m, 1H), 1.84-1.92 (m, 4H), 2.51 (dd, J=9.6, 7.2 Hz, 2H), 4.31 (m, 1H), 4.39 (dd, J=10.8, 6.0 Hz, 1H), 4.44-4.52 (m, 4H), 4.56-4.59 (m, 2H), 4.68 (dd, J=9.6, 3.6 Hz, 1H), 4.75 (m, 1H), 5.48 (d, J=3.6 Hz, 1H), 8.60 (d, J=8.8 Hz, 1H). HRMS calcd for C₄₂H₈₃NO₈ [M+Na]⁺752.6014, found: 752.5940.

### EXAMPLE 3

### Preparation of α-O-galactosyl Ceramide 1

The synthesis of KRN7000 was accomplished using the same strategy as that for 6. Phytosphingosine (Chiu, H.-Y. et al., J. Org. Chem. 68:5788-5791 (2003)) was converted into an activated acceptor (7) by amine to azide conversion and incorporation of PMB ethers on both of the secondary alcohols (Scheme 3). Glycosidation with donor 4 afforded the glycoconjugate (8) in 90% yield after column chromatography. Reduction of the azide, amidation, and global deprotection afforded KRN7000.

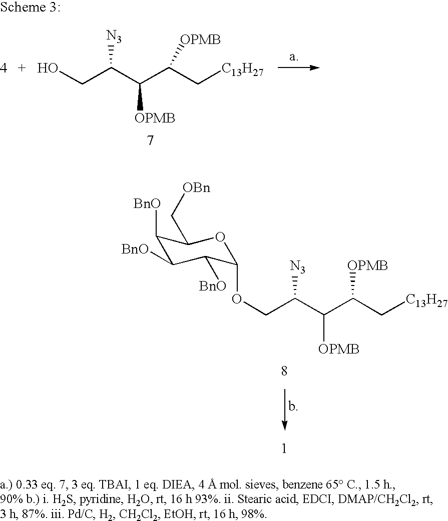

(2S,3S,4R)-2-Azido-3,4-di-para-methoxybenzyl-octadecane-1-ol (7). 7 was produced from the 2-azido-phytosphingosine (167 mg, 0.29 mmol) as slightly yellow oil in the same manner as described for 3. Yield: 161 mg (57% from the 2-azido-phytosphingosine). [α]_{D}²⁵−6.0° (C=0.5, CH₂Cl₂). ¹H NMR (400 MHz CDCl₃) δ 0.88 (t, J=6.8 Hz, 3H), 1.26-1.71 (m, 26H), 2.72 (t, J=6.4 Hz, 1H), 3.59-3.64 (m, 2H), 3.67 (dd, J=9.2, 4.4 Hz, 1H), 3.75-3.79 (m, 7H), 3.85 (dd, J=11.6, 5.6 Hz, 1H), 4.49 (d, J=11.2 Hz, 1H), 4.55 (d, J=11.2 Hz, 1H), 4.59 (d, J=11.2 Hz, 1H), 6.43 (d, J=11.2 Hz, 1H), 6.87 (dd, J=8.8, 1.6 Hz, 4H), 7.24 (dd, J=8.8, 2.8 Hz, 4H). ¹³C NMR (100 MHz, CDCl₃), δ 14.4, 22.9, 25.7, 29.6, 29.8, 29.8, 29.85, 29.9, 29.94, 30.4, 32.1, 55.4, 62.4, 63.2, 72.4, 73.4, 78.8, 80.4, 113.9, 114.0, 114.1, 129.6, 129.9, 130.0, 130.1, 130.3, 159.6, 159.7. ESIMS calcd for C₃₄H₅₃N₃O₅ [M+Na]⁺606.39, found: 606.50.

(2S,3S,4R)-2-Azido-3,4-di-O-para-methoxybenzyl-1-O-(2,3,4,6-tetra-O-benzyl-α-D-galactopyranosyl)-octadecane) (8). 8 was produced from 7 as clear oil (38 mg, 0.1 mmol) in the same manner as that of 5. Yield: 100 mg (90%). [α]_{D}²⁵+42.1° (C=0.65, CH₂Cl₂); ¹H NMR (400 MHz, CDCl₃) δ 0.88 (t, J=6.8 Hz, 3H), 1.26-1.39 (m, 26H), 3.49 (m, 2H), 3.55 (m, 1H), 3.69 (m, 2H), 3.75-3.80 (m, 7H), 3.94-4.00 (m, 4H), 4.06 (dd, J=9.6, 3.6 Hz, 1H), 4.35 (d, J=11.6 Hz, 1H), 4.38 (d, J=10.4 Hz, 1H), 4.44 (d, J=12.8 Hz, 1H), 4.70 (d, J=11.6 Hz, 1H), 4.51-4.58 (m, 3H), 4.69 (d, J=11.6 Hz, 1H), 4.73 (d, J=11.6 Hz, 1H), 4.80 (d, J=12.0 Hz, 1H), 4.82 (d, J=12.0 Hz, 1H), 4.90 (d, J=3.6 Hz, 1H), 4.94 (d, J=11.2 Hz, 1H), 6.83 (dd, J=8.8, 4.8 Hz, 4H) 7.20-7.37 (m, 24H). ¹³C NMR (100 MHz, CDCl₃) δ 14.4, 22.9, 25.6, 29.7, 29.9, 30.0, 30.1, 30.2, 32.2, 55.5, 62.3, 68.8, 69.2, 69.9, 71.9, 73.3, 73.4, 73.6, 73.7, 75.0, 75.3, 76.6, 78.8, 79.0, 79.1, 98.9, 114.0, 127.67, 127.7, 127.73, 127.8, 127.9, 128.97, 128.0, 128.46, 128.49, 128.6, 129.8, 129.84, 130.5, 130.7, 138.2, 138.3, 139.0, 139.1, 159.4, 159.5. ESIMS calcd for C₆₈H₈₇N₃O₃ [M+Na]+1128.63, found: 1128.43.

(2S,3S,4R)-2-Amino-3,4-di-O-para-methoxybenzyl-1-O-(2,3,4,6-tetra-O-benzyl-α-D-galactopyranosyl)-octadecane (Amino derivative). This product is produced from 8 (52 mg, 0.047 mmol) as white powder in the same manner as described above for the amino derivative of 1. Yield: 47 mg (93%). [α]_{D}²⁵+39.9° (C=1, CH₂Cl₂). ¹H NMR (400 MHz, CDCl₃) δ 0.88 (t, J=6.8 Hz, 3H), 1.26-1.62 (m, 26H), 3.16 (m, 1H), 3.37 (t, J=9.2 Hz, 1H), 3.45-3.55 (m, 3H), 3.64 (dt, J=9.6, 3.6 Hz, 1H), 3.74-3.81 (m, 7H), 3.92-3.98 (m, 3H), 4.04 (dd, J=11.6, 3.6 Hz, 1H), 4.35 (d, J=12.0 Hz, 1H), 4.41 (d, J=11.2 Hz, 1H), 4.43 (d, J=12.0 Hz, 1H), 4.46 (d, J=11.2 Hz, 1H), 4.52 (d, J=11.2 Hz, 1H), 4.56 (d, J=11.2 Hz, 1H), 4.61 (d, J=11.2 Hz, 1H), 4.65 (d, J=11.2 Hz, 1H), 4.73 (d, J=11.6 Hz, 1H), 4.79 (d, J=11.6, 1H), 4.80 (d, J=11.6, 1H), 4.89 (d, J=3.6 Hz, 1H), 4.93 (d, J=11.6 Hz, 1H), 6.83 (dd, J=8.8, 4.8 Hz, 4H) 7.20-7.37 (m, 24H). ¹³C NMR (100 MHz, CDCl₃) δ 14.4, 22.9, 26.0, 29.6, 29.9, 30.0, 30.1, 30.7, 32.2, 53.0, 55.5, 69.1, 69.6, 71.8, 71.9, 73.1, 73.4, 73.6, 73.7, 75.0, 75.1, 79.4, 79.7, 81.5, 99.2, 114.0, 127.6, 127.7, 127.8, 127.83, 127.9, 128.0, 128.1, 128.4, 128.5, 128.6, 129.7, 130.90, 131.0, 138.2, 138.8, 138.9, 139.0, 159.3, 159.4. ESIMS calcd for C₆₈H₈₉NO₁₀ [M+H]⁺ 1080.66, found: 1080.73.

(2S,3S,4R)-3,4-di-O-para-methoxybenzyl-1-O-(2,3,4,6-tetra-O-benzyl-α-D-galactopyranosyl)-2-(N-octacosylamino)-4-octadecane (Amide derivative). This compound was produced from the amino derivative (40 mg, 0.037 mmol) as a white powder in the same manner as described above for the amide derivative of 1. Yield: 44 mg (87%). [α]_{D}²⁵+25.9° (C=1.0, CH₂Cl₂). ¹H NMR (400 MHz, CDCl₃) δ 0.87 (t, J=6.8 Hz, 6H), 1.22-1.62 (m, 56H), 1.92 (m, 2H), 3.40-3.51 (m, 3H), 3.68-3.72 (m, 1H), 3.74 (s, 3H), 3.77 (s, 3H), 3.81 (dd, J=6.8, 2.8 Hz, 1H), 3.88-3.94 (m, 3H), 3.98 (dd, J=10.8, 5.2 Hz, 1H), 4.05 (dd, J=9.6, 3.6 Hz, 1H), 4.14 (m, 1H), 4.33 (d, J=11.2 Hz, 1H), 4.36 (d, J=13.2 Hz, 1H), 4.44 (d, J=12.8 Hz, 1H), 4.47 (d, J=13.2 Hz, 1H), 4.51 (d, J=12.8 Hz, 1H), 4.53 (d, J=11.2 Hz, 1H), 4.56 (d, J=11.6 Hz, 1H), 4.65 (d, J=12.0 Hz, 1H), 4.68 (d, J=11.2 Hz, 1H), 4.73 (d, J=12 Hz, 1H), 4.80 (d, J=12.0 Hz, 1H), 4.85 (d, J=3.6 Hz, 1H), 4.92 (d, J=12.0 Hz, 1H), 6.12 (d, J=8.8 Hz, 1H) 6.81 (t, J=8.8 Hz), 7.20-7.35 (m, 24H). ¹³C NMR (100 MHz, CDCl₃) δ 14.4, 22.9, 25.9, 26.3, 29.6, 29.64, 29.7, 29.8, 29.9, 30.0, 30.05, 30.1, 32.2, 36.9, 50.5, 55.5, 69.5, 69.6, 70.2, 71.5, 73.1, 73.4, 73.7, 73.8, 74.9, 75.0, 78.4, 79.2, 79.9, 99.7, 113.9, 114.0, 127.7, 127.8, 127.89, 127.94, 128.0, 128.1, 128.2, 128.4, 128.5, 128.53, 128.6, 128.7, 129.7, 129.9, 131.0, 137.8, 138.7, 138.9, 159.4, 173.0. ESIMS calcd for C₈₆H₁₂₃NO₁₁ [M+Na]⁺1368.90, found: 1369.31.

(2S,3S,4R)-1-O-(α-D-Galactopyranosyl)-2-(N-octadecanosylamino)-1,3,4-octadecenetriol (1). 1 was produced from the amide derivative (40 mg, 0.030 mmol) as a slightly yellow powder in the same manner as described for 6. Yield: 22 mg (98%). [α]_{D}²⁵+31.8° (C=1, pyridine). ¹H NMR (400 MHz, C₅D₅N) δ 0.88 (t, J=6.8 Hz, 6H), 1.25-1.34 (m, 50H), 1.68 (m, 1H), 1.82 (m, 2H), 1.88 (m, 2H), 2.35 (m, 1H), 2.46 (t, J=7.2 Hz, 2H), 4.35 (m, 2H), 4.40-4.48 (m, 4H), 4.53-4.58 (m, 2H), 4.65-4.70 (m, 2H), 5.30 (m, 1H), 5.61 (d, J=3.6 Hz, 1H), 8.54 (d, J=9.2 Hz, 1H). ¹³C NMR (100 MHz, C₅D₅N) δ 14.6, 23.3, 26.7, 26.9, 30.0, 30.1, 30.2, 30.23, 30.3, 30.4, 30.5, 30.7, 32.5, 34.7, 37.2, 51.9, 63.1, 69.1, 70.7, 71.4, 71.9, 72.9, 73.4, 77.1, 101.9, 173.6. HRMS calcd for C₄₂H₈₃NO₉ [M+Na]⁺768.5965, found: 768.5839.

### EXAMPLE 4

### Preparation of α-O-galactosyl ceramide 4,5-dehydro analog

Reaction of per-O-trimethylsilylgalactopyranose (9) with TMSI provided the galactosyl iodide in 10 min at 0° C. After evaporation of the solvent, 0.33 eq. of 3, 2 eq. TBAI, and 1.5 eq. DIEA in benzene were added to the iodide and stirred for 45 min. after which TLC indicated the reaction was complete. The crude product was refluxed in methanol with Dowex resin H+ for 45 min. The NMR spectrum of the resulting glycolipid (10) was clean and only the α anomer was observed. Preparation of α-O-galactosyl ceramide analogs can then proceed from the azide 11 following removal of the PMB protecting group using procedures described above, reduction of the azide using procedures described above, and condensation with a fatty acid of interest using the procedures described above.

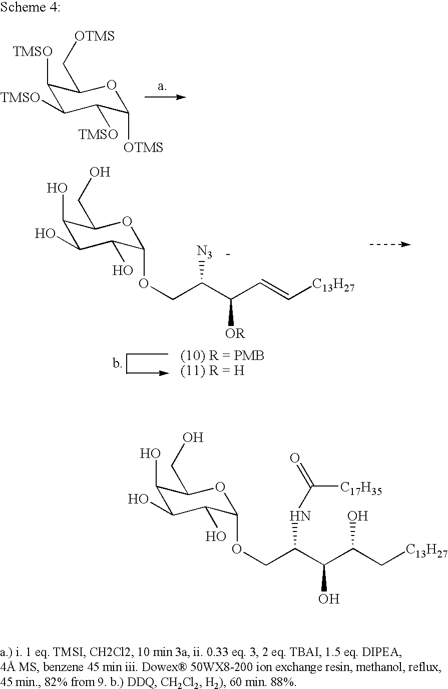

(2S,3R,4E)-2-Azido-3-O-para-methoxybenzyl-1-O-D-galactopyranosyl-4-octadecene (10). To a solution of 1,2,3,4,6-penta-O-trimethylsilyl-D-galactopyranose (160 mg, 0.30 mmol) in CH₂Cl₂ (2.5 mL) at 0° C. was added TMSI (60 mg, 0.30 mmol). After stirring at 0° C. for 10 min., the reaction was stopped by adding 5 mL of anhydrous toluene and azeotroped three times with toluene. The slightly yellow residue was dissolved in benzene (1 mL) and kept under argon. In a separate flask, molecular sieves (MS, 4 Å, 100 mg), TBAI (220 mg, 0.60 mmol), 3 (44.5 mg, 0.1 mmol) and DIPEA (58 mg, 0.45 mmol) was added into benzene (1 mL). The mixture was stirred under argon at 65° C. for 10 min. Upon dissolve of TBAI, the glycosyl iodide was cannulated into and the reaction mixture was stirred at 65° C. for 45 min. The reaction was stopped by adding EtOAc (10 mL) and cooled to 0° C. The white precipitate and MS was removed by filtration through Celite. The filtrate was washed with sat. Na₂S₂O₃ (aq) (2×10 mL) and brine, dried with anhydrous Na₂SO₄, concentrated in vacuo and the resulting residue was dissolved in MeOH (3 mL). Dowex® 50WX8-200 ion exchange resin (2 g) was added and the reaction was refluxed for 45 min. The resin was removed by filtration. The solvent was removed in vacuo and the resulting residue was purified by FCC to afford 10 (50 mg, 82% from 3) as clear oil. [α]_{D}²⁵+21° (C=1.0, CH₂Cl₂). ¹H NMR (400 MHz, CDCl₃) δ 0.87 (t, J=6.8 Hz, 3H), 1.24-1.40 (m, 22H), 1.67 (s, 2H), 2.10 (dd, J=13.6, 6.8 Hz, 2H), 2.29 (s, 1H), 2.55 (s, 1H), 3.00 (s, 1H), 3.20 (s, 1H), 3.49-3.52 (m, 1H), 3.57 (dd, J=10.4, 6.8 Hz, 1H), 3.70 (dd, J=10.4, 6.8 Hz, 1H), 3.73-3.83 (m, 7H), m (2H), 4.07 (d, J=2.8 Hz, 1H), 4.25 (d, J=11.6 Hz, 1H), 4.51 (d, J=11.6 Hz, 1H), 4.91 (d, J=3.6 Hz, 1H), 5.37 (dd, J=15.6, 8.4 Hz, 1H), 5.74 (dt, J=15.6, 6.8 Hz, 1H), 6.86 (d, J=8.8 Hz, 2H), 7.21 (d, J=8.8 Hz, 2H). ¹³C NMR (100 MHz, CDCl₃) δ 14.3, 22.9, 29.2, 29.5, 29.6, 29.7, 29.8, 29.9, 32.1, 32.6, 55.4, 62.3, 64.7, 68.4, 69.2, 69.8, 70.2, 70.7, 79.3, 99.9, 114.0, 126.0, 129.6, 130.1, 138.5, 159.4. ESIMS calcd for C₃₂H₅₃N₃O₈ [M+Na]⁺630.38, found: 630.69.

(2S,3R,4E)-2-Azido-3-hydroxy-1-O-α-D-galactopyranosyl-4-octadecene (11). To a solution of 10 (50 mg, 0.082 mmol) in CH₂Cl₂: H₂O (20:1) was added DDQ (28 mg, 0.123 mmol). The reaction mixture was stirred under argon for 60 min. Sat. NaHCO₃ (5 mL) was added and the solution was extracted with CH₂C₂ (5×10 mL). The organic layer was combined and dried with anhydrous Na₂SO₄. The solvent was evaporated ill vacuo and the resulting residue was purified by FCC to afford 11 (35 mg, 88%) as a white powder. [α]_{D}²⁵+66° (C=0.90, MeOH). ¹H NMR (400 MHz, CD₃OD) δ 0.89 (t, J=6.8 Hz, 3H), 1.28-1.42 (m, 22H), 2.07 (dd, J=13.6, 6.8 Hz, 2H), 3.52 (dt, J=9.2, 2.8 Hz, 1H), 3.55 (dd, J=10.8, 6.8 Hz, 1H), 3.69 (d, J=6.4 Hz, 1H), 3.70 (d, J=6.4 Hz, 1H), 3.76 (d, J=5.6 Hz, 1H), 3.77 (d, J=5.6 Hz, 1H), 3.84 (t, J=6.8 Hz, 1H), 3.88-3.91 (m, 2H), 4.19 (t, J=6.8 Hz, 1H), 4.84 (d, J=3.6 Hz, 1H), 5.52 (dd, J=15.2, 7.6 Hz, 1H), 5.75 (dt, J=15.2, 6.8 Hz, 1H). ¹³C NMR (100 MHz, CDCl₃) δ 14.4, 22.9, 29.3, 29.6, 29.7, 29.8, 29.9, 30.0, 32.2, 32.7, 62.2, 65.1, 68.1, 68.9, 70.4, 71.9, 99.6, 128.6, 135.7. ESIMS calcd for C₂₄H₄₅N₃O₇ [M+Na]⁺510.32, found: 510.54.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method of producing an α-O-galactosyl ceramide precursor comprising the step of contacting a galactosyl iodide with a quaternary ammonium iodide salt and a sphingosine derivative or a phytosphingosine derivative under conditions sufficient to produce an α-O-galactosyl ceramide precursor.

2. The method of claim 1, wherein said galactosyl iodide has the following formula: 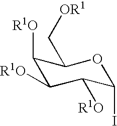
wherein each R¹ is an independently selected protecting group.

3. The method of claim 2, wherein each R¹ is a benzyl group.

4. The method of claim 2, wherein each R¹ is a tri-methyl silyl group.

5. The method of claim 1, wherein said quaternary ammonium iodide salt is tetra-butylammonium iodide.

6. The method of claim 1, wherein said sphingosine derivative has the following formula: 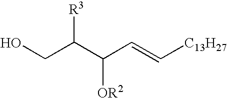
wherein
R² is a member selected from the group consisting of a protecting group and H; and
R³ is a member selected from the group consisting of an amine, an amide and an azide.

7. The method of claim 6, wherein R² is a para-methoxybenzyl group.

8. The method of claim 6, wherein R² is tri-methyl silyl group.

9. The method of claim 6, wherein R³ is an azide.

10. The method of claim 1, wherein said phytosphingosine derivative has the following formula: 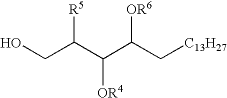
wherein
R⁴ and R⁶ are each independently a member selected from the group consisting of a protecting group and H; and
R⁵ is a member selected from the group consisting of an amine, an amide and an azide.

11. The method of claim 10, wherein R⁴ and R⁶ are each para-methoxybenzyl.

12. The method of claim 10, wherein R⁵ is an azide.

13. The method of claim 1, wherein said α-O-galactosyl ceramide precursor is prepared in at least 80% yield.

14. A method of producing an α-O-galactosyl ceramide comprising the following steps:
a) contacting a galactosyl iodide with a quaternary ammonium iodide salt and a sphingosine derivative or a phytosphingosine derivative under conditions sufficient to produce an α-O-galactosyl ceramide precursor; and
b) contacting said α-O-galactosyl ceramide precursor with a fatty acid or fatty acid derivative under conditions appropriate to produce an α-O-galactosyl ceramide.

15. The method of claim 14, wherein said galactosyl iodide has the following formula: 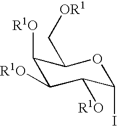
wherein each R¹ is an independently selected protecting group.

16. The method of claim 15, wherein each R¹ is a benzyl group or a tri-methyl silyl group.

17. The method of claim 14, wherein said quaternary ammonium iodide salt is tetra-butylammonium iodide.

18. The method of claim 14, wherein said sphingosine derivative has the following formula: 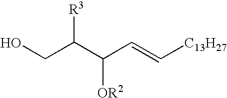
wherein
R² is a para-methoxybenzyl group or a tri-methyl silyl group; and
R³ is an azide.

19. The method of claim 14, wherein said phytosphingosine derivative has the following formula: 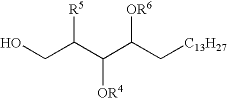
wherein
R⁴ and R⁶ are each para-methoxybenzyl; and
R⁵ is an azide.

20. The method of claim 14, wherein said fatty acid is stearic acid.
